SEQUENCE LISTING (4 sequences)

SEQ ID NO: 1 - DNA, 30 bp, Homo sapiens
gatatcctaacaccagctgacgagaaactg

SEQ ID NO: 2 - DNA, 27 bp, Homo sapiens
gatatctgcacaccccggccgtcccag

SEQ ID NO: 3 - DNA, 36 bp, Homo sapiens
tctagagttttcaaatttgaattttaatgtgtgttg

SEQ ID NO: 4 - DNA, 36 bp, Homo sapiens
ggatcccaccttaaggaggttgcaacgagcgtggca

(19) United States
(12) Patent Application Publication
(10) Pub. No.: US 2016/0346363 A1
(43) Pub. Date: Dec. 1, 2016

(54) METHODS AND ORAL FORMULATIONS FOR ENZYME REPLACEMENT THERAPY OF HUMAN LYSOSOMAL AND METABOLIC DISEASES

(71) Applicant: NEW YORK UNIVERSITY, New York, NY (US)

(72) Inventors: Kam-meng TCHOU-WONG, New York, NY (US); Frank MARTINIUK, Woodridge, NJ (US)

(21) Appl. No.: 14/905,114

(22) PCT Filed: Jul. 14, 2014
(86) PCT No.: PCT/US2014/046469

§ 371 Date: Jan. 14, 2016

Related U.S. Application Data
(60) Provisional application No. 61/846,135, filed on Jul. 15, 2013.

(51) Int. Cl. A61K 38/47; A61K 45/06; C12N 9/26; A61K 9/00

(52) U.S. Cl. CPC A61K 38/47; A61K 9/0053; C12Y 302/0102; A61K 45/06; C12N 9/2408

(57) ABSTRACT

The invention provides methods, compositions and kits for enzyme replacement therapy as well as molecular constructs, cells, tissues and plants suitable for expressing recombinant enzymes. Similarly, the invention provides methods for recombinantly producing and orally administering certain metabolic or lysosomal enzymes such as acid alpha glucosidase (GAA) alone, in a pharmaceutical composition or with an activator protein (AGA). Also, the invention provides methods for treating a glycogen storage disease type II (GSDII) or acid maltase deficiency (AMD) or Pompe disease or Fabry disease.

### STATEMENT OF GOVERNMENT RIGHTS

The present invention was developed, at least in part, using government support under Contract No. UL1 TR000038 awarded by the National Center for Advancing Translational Sciences, National Institutes of Health. Therefore, the Federal Government may have certain rights in the invention.

### FIELD OF THE INVENTION

The present invention relates to methods, compositions and kits for oral enzyme replacement therapy as well as plants, seeds and molecular constructs suitable for expressing recombinant enzymes.

### BACKGROUND OF THE INVENTION

### Transgenic Plants, Seeds and Cultured Plant Cells

Transgenic plants, seeds and cultured plant cells are potentially one of the most economical systems for large-scale production of recombinant enzymes for pharmaceutical uses (Kermode, Can J Bot., 2006; 84: 679-694; Kermode, Seed Expression Systems for Molecular Farming. In: Wang, A., Ma, S. (eds) Molecular farming in plants: recent advances and future prospects. Springer, New York, 2012; pp 89-123; Lau, et al., Biotechnol Adv., 2009; 27: 1015-1022). Seeds are particularly attractive for use due to their high rates of protein synthesis and their ability to remain viable in a mature-dry state (Twyman, et al., Trends Biotechnol., 2003; 21: 570-578; Boothe, et al., Plant Biotechnol J., 2010; 8: 588-606; Stoger, et al., Curr Opin Biotechnol., 2005; 16: 167-173). Over one-third of approved pharmaceutical proteins are glycoproteins (Gomord, et al., Plant Biotechnol J., 2010; 8: 564-587; Saint-Jore-Dupas, et al., Trends Biotechnol., 2007; 25: 317-323) and even minor differences in N-glycan structures can change the distribution, activity or longevity of recombinant proteins compared to their native counterparts thereby altering their efficacy as therapeutics. Thus, one of the major challenges of using plants as systems for pharmaceutical glycoprotein production is producing these pharmaceuticals with humanized N-glycans. Notably, certain processes of N-glycosylation that occur after proteins leave the post-endoplasmic reticulum along the secretory pathway are markedly different in plant cells versus mammalian cells. On the other hand, early steps and components of the N-glycosylation process in the endoplasmic reticulum (including the involvement of the dolichol lipid intermediate and endoplasmic reticulum oligosaccharide transferase) and the Golgi-localized N-acetylglucosaminyl transferase I are similar in plant and mammalian cells (Lerouge, et al., Plant Mol Biol., 1998; 38: 31-48). For example, in the plant Golgi complex, enzymes convert the original high-mannose N-glycans of proteins to plant-specific hybrid and complex N-glycans by a series of sequential reactions that rely on the accessibility of the glycan chain(s) to the Golgi processing machinery (Gomord, et al., Curr Opin Plant Biol., 2004; 7: 171-181; Kermode, Crit Rev Plant Sci., 1996; 15: 285-423). Plant-specific sugars that are associated with these “matured” N-glycans, such as β-1,2-xylose and α-1,3-fucose, may induce immune responses in humans, particularly when parenterally administrated (Gomord, et al., Plant Biotechnol J., 2010; 8: 564-587; He, et al., Glycobiology, 2012; 22: 492-503).

Many studies in animal models are evaluating oral formulations or edible tissues in rice, corn-maize, tobacco, broccoli sprouts, tomato or pea for delivery, immunization, vaccination and prevention of gastrointestinal infections, pollen allergies, diabetes, endocrine-associated diseases including growth hormone insensitivity syndrome, hypersensitivity, bacterial infection, elevated blood pressure, cholera, viral infection, leucopenia, asthma, apoptosis, prostate cancer, rheumatoid arthritis, cytokines and to induce effective mucosal immune tolerance and immune reactions (Zimmermann, et al., BMC Biotechnology, 2009; 9: 79-101; Pena-Ramirez, et al., Clinical and Vaccine Immunology, 2007; 14: 685-692; Takagia, et al., Peptides, 2010; 31: 1421-1425; Xie, et al., Peptides., 2008; 29: 1862-1870; Yamada, et al., Peptides, 2008; 29: 331-337; Lamphear, et al., Journal Controlled Release, 2002; 85: 169-180; Yang, et al., FEBS Letters, 2006; 580: 3315-3320; Takagi, et al., Vaccine, 2008; 26: 6027-6030; Streatfield, et al., Vaccine, 2003; 21: 812-815; Yang, et al., Biochemical and Biophysical Research Communications, 2008; 365: 334-339; Cheung, et al., BMC Biotechnology, 2011; 11: 37-14; Takagi, et al., PNAS, 2005; 102: 17525-17530; Takagi, et al., Plant Biotechnology Journal, 2005; 3: 521-533; Wu, et al., Plant Biotechnology Journal, 2007; 5: 570-578; Ning, et al., Biotechnol Lett., 2008; 30: 1679-1686; Yang, et al., J AOAC International, 2008; 91: 957-66; Suzuki, et al., Int Arch allergy Immunol., 2009; 149(suppl 1): 21-24; Suzuki, et al., Plant Biotechnology Journal, 2011; 9: 982-990; Tackaberry, et al., Vaccine, 1999; 17: 3020-9; Keum, et al, Pharmaceutical Research; 2009; 26: 2324-2331; Hashizume, et al., Transgenic Res., 2008; 17: 1117-1129; Sardana, et al., Transgenic Research, 2002; 11: 521-531; Takaiwa, et al., Human Vaccines, 2011; 7: 357-366; Takaiwa, Immunotherapy, 2013; 5:301-12). However, only one report has proposed or demonstrated oral delivery of bioactive enzymes for enzyme replacement therapy of diseases deficient in metabolic or lysosomal enzymes or proteins.

### Lysosomal Storage Diseases

A malfunction of a specific acid hydrolase leads to accumulation of the substrate in lysosomes, leading to a variety of pathologies including Tay-Sachs disease, due to a deficiency of the enzyme β-N-hexosaminidase, Mucopolysaccharidoses (MPSs), a group of recessive disorders due to a malfunction in the degradation of complex sulphurates, Anderson-Fabry disease due to a deficiency of α-galactosidase A causing accumulation of globotriaosylceramide in renal microvascular endothelial cells, Pompe disease due to a deficiency of acid α-glucosidase leading to intralysosomal accumulation of glycogen, and Gaucher disease due to a deficiency in β-glucosidase causing accumulation of glycosphingolipids mainly in cells of monocyte-macrophage lines.

Human lysosomal enzymes can be produced in transgenic plants in order to solve problems of safety, viral infections, immune reactions, production yield and cost. Radin et al., U.S. Pat. No. 5,929,304 teaches in-leaf production of some lysosomal enzymes (glucocerebrosidase and α-L-iduronidase) in tobacco. The enzymes are produced essentially in the leaves by plants transformed via the use of vectors containing the MeGa promoter (from the tomato HMG2 promoter) or the cauliflower mosaic virus (CaMV) 35S promoter

### Acid Alpha Glucosidase (GAA) and Pompe Disease

Acid maltase or acid alpha glucosidase (GAA) is a lysosomal enzyme that hydrolyzes glycogen to glucose (Hers, Biochem J., 1963; 86: 11-16). The enzyme is synthesized and processed via a pathway common to lysosomal enzymes (Kornfeld, J Clin Invest., 1986; 77: 1-6; Rosenfeld, et al., J Cell Biol., 1982; 93: 135-141). The native protein is initially synthesized as an approximately 120 kD monomer and undergoes further trimming into two major bands of 80 and 70 kD and smaller sized bands when analyzed on SDS-PAGE (Oude Elferink, PhD Thesis, University of Amsterdam, 1985 Biosynthesis, transport and processing of lysosomal alpha glucosidase). Genetic deficiency of acid alpha glucosidase results in glycogen storage disease type II (GSDII) or acid maltase deficiency (AMD) or Pompe disease, encompassing at least five clinical subtypes of varying severity (infantile, non-classical infantile, childhood, juvenile and late onset) (Slonim, et al., J Pediatrics, 2000; 137: 283-5). Pompe disease is a lysosomal storage disease caused by deficiency in acid alpha glucosidase (GAA) activity that leads to the accumulation of glycogen in tissues (primarily muscle) and is characterized by progressive skeletal muscle weakness and respiratory insufficiency. Pompe disease is clinically heterogeneous in the age of onset, the extent of organ involvement, and the rate of progression. The infantile form presents as hypotonia, muscle weakness and congestive heart failure in the first year. The childhood and juvenile forms are fatal by the second decade of life, while the later onset forms are limited to skeletal muscle.

The current enzyme replacement therapy for Pompe disease is by intravenous infusion of Myozyme/Lumizyme (a recombinant human acid alpha glucosidase (GAA) produced by Chinese Hamster Ovary (CHO) cells) once every two weeks. In contrast to the single bolus infusion of a high dose of Myozyme every two weeks, it is desirable to improve the efficacy of enzyme replacement therapy of Pompe disease by administering an acid alpha glucosidase enzyme in combination with its activator protein (AGA) daily and orally administering the enzyme replacement therapy (Oral-ERT) thereby allowing maintenance of a therapeutic dose of enzyme activity on a daily basis in the patients. Currently, there is no effective treatment or cure for GSDII. Enzyme and gene replacement therapies are being developed.

Enzyme replacement therapy (ERT) treatment for Pompe disease includes two approved products based on the intravenous administration of recombinant human GAA produced in a Chinese Hamster Ovary (CHO) cell line, Myozyme and Lumizyme (alglusidase alfa, Genzyme Corporation, Cambridge, Mass.). Enzyme replacement therapy has shown varying efficacy in patients using a biweekly infusion regimen. In late-onset patients, mild improvements in motor and respiratory functions have been achieved after enzyme replacement therapy but long-term evaluation will be needed to confirm clinical efficacy (Strothotte, et al., J Neurol., 2010; 257: 91-97; van der Ploeg, et al., N Engl J Med., 2010; 362: 1396-1406). A number of reports show that correction of the skeletal muscle phenotype is challenging, and not all patients respond equally well to treatment (van der Ploeg, et al., Lancet, 2008; 372: 1342-1353; Van den Hout, et al., Pediatrics, 2004; 113: e448-457; Thurberg, et al., Lab Invest., 2006; 86: 1208-1220; Schoser, et al., Neurotherapeutics, 2008; 5: 569-578). The challenges for enzyme replacement therapy for Pompe disease include insufficient targeting/uptake of enzyme into disease-relevant tissues and poor tolerability due to severe ERT-mediated anaphylactic and immunologic reactions (van der Ploeg, et al., N Engl J Med., 2010; 362: 1396-1406; Kishnani, et al., J Pediatr., 2006; 149: 80-97; Raben, et al., Mol Genet Metab., 2003; 80: 159-169; Fukuda, et al., Autophagy, 2006; 2: 318-320; Cardone, et al., Pathogenetics, 2008; 1: 6-28; Kishnani, et al., Mol GenetMetab., 2010; 99: 26-33; de Vries, et al., Mol GenetMetab., 2010; 338-345).

Since enzyme replacement therapy (ERT) of Pompe disease has only been administered by a single infusion of enzyme once every two weeks, the bolus infusion of enzyme only provides a transient, high level of enzyme which decreases over the course of the remaining two weeks. Oral administration (Oral-ERT) would allow more frequent administration at more frequent intervals to maintain a therapeutic dose of enzyme on a daily basis.

### Acid α-Galactosidase A (GALA) and Fabry Disease

The α-galactosidase A (GALA) gene encodes a homodimeric glycoprotein that hydrolyses the terminal α-galactosyl moieties from glycolipids and glycoproteins. This enzyme predominantly hydrolyzes ceramide trihexoside and melibiose into galactose and glucose. Mutations in GALA affect the synthesis, processing and stability of the enzyme in Fabry disease, a rare X-linked lysosomal storage disorder that can cause a wide range of systemic symptoms. The incidence of Fabry disease is estimated to be between 1 in 40,000 and 1 in 120,000 live births. Patients with Fabry disease may experience a wide range of signs and symptoms including kidney failure, heart problems and stroke. Full body or localized pain to the extremities (acroparesthesia) or the gastrointenstinal tract is common. Angiokeratomas (tiny, painless papules that may appear on any region of the body, but are predominant on the thighs, around the belly-button, buttocks, lower abdomen, and groin) are a common symptom. Anhidrosis (lack of sweating) is a common symptom and less commonly hyperhidrosis (excessive sweating).

### Expression of Lysosomal Enzymes in Plants

Fogher, et al., EP1480510 teach expression of lysosomal enzymes in plant seeds. Fogher et al., U.S. Patent Publication 2006/0031965 teach in-seed expression of lysosomal enzymes and transgenic plants able to express the lysosomal enzymes in seed storage tissues in enzymatically active form and in amounts appropriate for use in enzyme replacement therapy. Also, Martiniuk, U.S. Patent Publication 2001/0027250 teaches an activator protein of human acid maltase (AGA) and uses thereof.

Rossi, et al., Vet Res Commun., 2014; 38: 39-49 showed protective effect of oral administration of transgenic tobacco seeds expressing FedA against verocytotoxic Escherichia coli strain in piglets. Gorantala, et al., J Biotechnology, in press, 2014 generated protective immune response against anthrax by oral immunization with protective antigen tobacco and mustard-based vaccine. Yang, et al., FEBS Letters, 2006; 580: 3315-3320 produced a transgenic rice seed accumulating an anti-hypertensive peptide reduces the blood pressure of spontaneously hypertensive rats. Xie, et al., Peptides, 2008; 29: 1862-1870 developed a biologically active rhIGF-1 fusion accumulated in transgenic rice seeds can reduce blood glucose in diabetic mice via oral delivery. Ning, et al., Biotechnol Lett., 2008; 30: 1679-1686 showed that oral administration of recombinant human granulocyte macrophage colony stimulating factor expressed in rice endosperm can increase leukocytes in mice.

Shaaltiel, et al., Plant Biotechnology J., 2007; 5: 579-590 and U.S. Pat. No. 8,227,230 report producing a glucocerebrosidase with terminal mannose glycans for enzyme replacement therapy of Gaucher disease using a plant cell culture system, specifically carrot. A clinical trial is expected—Clinical Trials Identifier: NCT01747980—An Exploratory, Open-label Study to Evaluate the Safety of PRX-112 and Pharmacokinetics of Oral prGCD (Plant Recombinant Human Glucocerebrosidase) in Gaucher Patients receiving 250 ml of re-suspended carrot cells administered orally. Absorption of therapeutic proteins taken orally has remained a major hurdle for the treatment of diseases in humans. Proteins are generally degraded by enzymes in the stomach and intestine; additionally, the intestine lining can prevent absorption into the circulation. Administration of PRX-112, a plant recombinant human glucocerebrosidase using plant cells as carrier vehicle, may help overcome many of these hurdles. The plant cell wall protects the protein from degradation in its transport through the upper GI and allows release in the lower intestine. Studies in animals have shown that prGCD delivered in this way can be found in the blood stream in an active form (Grabowski, et al., Mol Gen Met., in press, 2014).

It would be desirable to provide genetically engineered seeds, such as tobacco seeds, as an alternative large-scale production system that overcomes the barrier of the high cost of producing recombinant human enzymes for effective enzyme replacement therapy. Several expression systems have been developed in plants that potentially offer many advantages in terms of production, scaling up, economy and safety of the therapeutic molecules (Fischer, et al., Biotechnol Adv., 2012; 30: 434-9; Lico, et al., Plant Cell Rep., 2012; 31: 439-51; Twyman, et al., Trends Biotechnol., 2003; 21: 570-578; Twyman, et al., Trends Biotechnol., 2009; 27: 609-12). The technological platform involving the accumulation of recombinant proteins in seeds warrants a better availability of the products and allows long-term storage of the biomass for processing (Kusnadi, et al., Biotechnol Bioeng., 1998; 60: 44-52; Reggi, et al., Plant Molecular Biology, 2005; 57: 101-113; Stoeger, et al., Plant Molecular Biology, 2000; 42: 583-590). Especially, it would be desirable to provide an enzyme replacement therapy with tobacco-produced recombinant human activator protein (tobrhAGA) singly or in combination with tobacco-produced recombinant human acid alpha glucosidase (tobrhGAA) for treating Pompe disease as well as formulations for orally administering tobhAGA singly or co-administering tobhAGA and tobrhGAA as, for instance, pill/gel/capsules or slow time-release formats or as added to milk or formulas or beverages or diet supplements.

### SUMMARY OF THE INVENTION

The present invention is based in part on the discovery of a single and combination oral-enzyme replacement (Oral-ERT) therapy featuring recombinant human acid alpha glucosidase (GAA) and its activator protein (AGA) in transgenic tobacco and non-tobacco plants and oral formulations for oral enzyme replacement therapy in pill, gel, or capsule form for safe, convenient daily, multiple administration by ingestion in contrast to the single intravenous infusion approximately every 2 weeks in accordance with currently available therapies. The present invention provides the benefit of allowing maintenance of a daily therapeutic level of acid alpha glucosidase (GAA) enzyme activity to improve quality of life as well as life span. Further, the present invention provides the benefits of reduced cost, safety and convenience compared to currently available alternatives.

In a first aspect, the invention provides methods for replacing a metabolic or lysosomal enzyme in a subject that is present or biologically active in a suboptimal or deficient amount in the subject by orally administering the metabolic or lysosomal enzyme or a fragment or a variant thereof or a pharmaceutical composition containing the metabolic or lysosomal enzyme or a fragment or a variant thereof or a plant extract containing the metabolic or lysosomal enzyme or a fragment or variant thereof to the subject. The metabolic or lysosomal enzyme or fragment or variant thereof may be produced recombinantly, such as, for instance in one or more isolated plant cells such as one or more plant seeds, or in a whole plant by any suitable recombinant constructs including those described herein. The plant cells or plant may be for instance, a tobacco plant or cell thereof or a tobacco seed.

The metabolic or lysosomal enzyme may be, for instance, acid alpha glucosidase (GAA) or α-galactosidase A (GALA). The metabolic or lysosomal enzyme may also be, for instance, one of α-N-acetylgalactosaminidase, acid lipase, aryl sulfatase A, aspartylglycosaminidase, ceramidase, α-fucosidase, β-galactosidase, galactosylceramidase, glucocerebrosidase, β-glucuronidase, heparin N-sulfatase, β-hexosaminidase, iduronate sulfatase, α-L-iduronidase, α-mannosidase, β-mannosidase, sialidase, and sphingomyelinase. The metabolic or lysosomal enzyme such as, for instance, acid alpha glucosidase (GAA) or α-galactosidase A (GALA), may be administered alone or in combination with one or more activator protein (AGA) of the same. The subject may be a mammal including a human, and the subject may be suffering from a glycogen storage disease type II (GSDII) or acid maltase deficiency (AMD) or Pompe disease or Fabry disease.

The metabolic or lysosomal enzyme or a pharmaceutical composition or plant extract containing the metabolic or lysosomal enzyme may be orally administered to the subject one, two, three, four, five, six, seven or more times per week, or one, two, three, or more times per day. Likewise, the metabolic or lysosomal enzyme or a pharmaceutical composition containing the metabolic or lysosomal enzyme may be orally administered in gel, pill, tablet, liquid or capsule form or added to milk or formulas or beverages or diet supplements.

Administering the metabolic or lysosomal enzyme to the subject may result in an increase in the biological activity or amount of the metabolic or lysosomal enzyme in the subject of about 10%, 20%, 30%, 50%, 75%, 100%, 200%, 300%, 400%, 500%, or ten times, fifteen times, twenty times or more within, for instance, about an hour, a few hours, a day, a few days, or a week.

In a second aspect, the invention provides methods of treating a disease caused by a deficiency of biological activity or amount of a metabolic or lysosomal enzyme by orally administering the metabolic or lysosomal enzyme or a fragment or a variant thereof or a pharmaceutical composition or plant extract containing the metabolic or lysosomal enzyme or a fragment or variant thereof to the subject. The metabolic or lysosomal enzyme or fragment or variant thereof may be produced recombinantly, such as, for instance in one or more isolated plant cells such as one or more plant seeds, or in a whole plant by any suitable recombinant constructs including those described herein. The plant cells or plant may be for instance, a tobacco plant or cell thereof or a tobacco seed.

The metabolic or lysosomal enzyme may be, for instance, acid alpha glucosidase (GAA) or α-galactosidase A (GALA). The metabolic or lysosomal enzyme may also be, for instance, one of α-N-acetylgalactosaminidase, acid lipase, aryl sulfatase A, aspartylglycosaminidase, ceramidase, α-fucosidase, β-galactosidase, galactosylceramidase, glucocerebrosidase, β-glucuronidase, heparin N-sulfatase, β-hexosaminidase, iduronate sulfatase, α-L-iduronidase, α-mannosidase, β-mannosidase, sialidase, and sphingomyelinase. The metabolic or lysosomal enzyme such as, for instance, acid alpha glucosidase (GAA) or α-galactosidase A (GALA), may be administered alone or in combination with one or more activator protein (AGA) of the same. The subject may be a mammal including a human, and the subject may be suffering from a glycogen storage disease type II (GSDII) or acid maltase deficiency (AMD) or Pompe disease or Fabry disease.

The metabolic or lysosomal enzyme or a pharmaceutical composition or plant extract containing the metabolic or lysosomal enzyme may be orally administered to the subject one, two, three, four, five, six, seven or more times per week, or one, two, three, or more times per day. Likewise, the metabolic or lysosomal enzyme or a pharmaceutical composition containing the metabolic or lysosomal enzyme may be orally administered in gel, pill, tablet, liquid or capsule form or added to milk or formulas or beverages or diet supplements.

Administering the metabolic or lysosomal enzyme to the subject may result in an increase in the biological activity or amount of the metabolic or lysosomal enzyme in the subject of about 10, 20%, 30%, 50%, 75%, 100%, 200%, 300%, 400%, 500%, or ten times, fifteen times, twenty times or more within, for instance, about an hour, a few hours, a day, a few days, or a week.

In a third aspect, the invention provides a pharmaceutical composition containing a metabolic or lysosomal enzyme or a fragment or a variant thereof and optionally an activator protein of the same. The metabolic or lysosomal enzyme or a fragment or a variant thereof or an activator protein of the same may be produced recombinantly, such as, for instance in one or more isolated plant cells such as one or more plant seeds, or in a whole plant by any suitable recombinant constructs including those described herein. The plant cells or plant may be for instance, a tobacco plant or cell thereof or a tobacco seed. The metabolic or lysosomal enzyme may be, for instance, acid alpha glucosidase (GAA) or α-galactosidase A (GALA). The metabolic or lysosomal enzyme may also be, for instance, one of α-N-acetylgalactosaminidase, acid lipase, aryl sulfatase A, aspartylglycosaminidase, ceramidase, α-fucosidase, β-galactosidase, galactosylceramidase, glucocerebrosidase, β-glucuronidase, heparin N-sulfatase, β-hexosaminidase, iduronate sulfatase, α-L-iduronidase, α-mannosidase, β-mannosidase, sialidase, and sphingomyelinase. The pharmaceutical composition may also contain one or more activator protein (AGA) of the metabolic or lysosomal enzyme, for instance, acid alpha glucosidase (GAA) or α-galactosidase A (GALA). The pharmaceutical composition may contain one or more isolated plant cells or one or more plant tissues. The plant may be, for instance, a tobacco plant. The pharmaceutical composition containing the metabolic or lysosomal enzyme may be designed for oral administration one, two, three, four, five, six, seven or more times per week, or one, two, three, or more times per day, and it may be designed for administration as a gel, a tablet, a liquid or a capsule form as well as designed for sustained release or added to milk or formulas or beverages or diet supplements.

In a fourth aspect, the invention provides a genetic construct such as a vector containing a nucleic acid sequence encoding a metabolic or lysosomal enzyme or a fragment or variant thereof. The nucleic acid sequence may be a cDNA and may encode acid alpha glucosidase (GAA) or α-galactosidase A (GALA). Also, the nucleic acid sequence may be a cDNA and may encode one of α-N-acetylgalactosaminidase, acid lipase, aryl sulfatase A, aspartylglycosaminidase, ceramidase, α-fucosidase, β-galactosidase, galactosylceramidase, glucocerebrosidase, β-glucuronidase, heparin N-sulfatase, β-hexosaminidase, iduronate sulfatase, α-L-iduronidase, α-mannosidase, β-mannosidase, sialidase, and sphingomyelinase. The genetic construct may also contain one or more nucleic acid sequences encoding an activator protein (AGA) of the metabolic or lysosomal enzyme, for instance, acid alpha glucosidase (GAA) or α-galactosidase A (GALA). The genetic construct containing a nucleic acid sequence encoding a metabolic or lysosomal enzyme or a fragment or a variant thereof may be suitable for or adapted for transfecting a plant cell or a plant tissue. The plant may be, for instance, a tobacco plant. The genetic construct may further contain one or more regulatory sequences, such as, for instance a promoter, enhancer or termination sequence, and may in some instances by adapted for transient or constitutive expression. An exemplary genetic construct is provided in FIG. 1.

In a fifth aspect, the invention provides a plant cell containing a genetic construct such as a vector containing a nucleic acid sequence encoding a metabolic or lysosomal enzyme or a fragment or variant thereof. The nucleic acid sequence may be a cDNA and may encode acid alpha glucosidase (GAA) or α-galactosidase A (GALA) or a fragment or a variant thereof. Also, the nucleic acid sequence may be a cDNA and may encode one of α-N-acetylgalactosaminidase, acid lipase, aryl sulfatase A, aspartylglycosaminidase, ceramidase, α-fucosidase, β-galactosidase, galactosylceramidase, glucocerebrosidase, β-glucuronidase, heparin N-sulfatase, β-hexosaminidase, iduronate sulfatase, α-L-iduronidase, α-mannosidase, β-mannosidase, sialidase, and sphingomyelinase. The genetic construct may also contain one or more nucleic acid sequences encoding an activator protein (AGA) of the metabolic or lysosomal enzyme, for instance, acid alpha glucosidase (GAA) or α-galactosidase A (GALA). The genetic construct containing a nucleic acid sequence encoding a metabolic or lysosomal enzyme or a fragment or variant thereof may be suitable for or adapted for transfecting a plant cell or a plant tissue. The plant may be, for instance, a tobacco plant. The genetic construct may further contain one or more regulatory sequences, such as, for instance a promoter, enhancer or termination sequence, and may in some instances by adapted for transient or constitutive expression. An exemplary genetic construct is provided in FIG. 1.

In a sixth aspect, the invention provides a recombinant plant containing a genetic construct such as a vector containing a nucleic acid sequence encoding a metabolic or lysosomal enzyme or a fragment or variant thereof. The nucleic acid sequence may be a cDNA and may encode acid alpha glucosidase (GAA) or α-galactosidase A (GALA) or a fragment or a variant thereof. Also, the nucleic acid sequence may be a cDNA and may encode one of α-N-acetylgalactosaminidase, acid lipase, aryl sulfatase A, aspartylglycosaminidase, ceramidase, α-fucosidase, β-galactosidase, galactosylceramidase, glucocerebrosidase, β-glucuronidase, heparin N-sulfatase, β-hexosaminidase, iduronate sulfatase, α-L-iduronidase, α-mannosidase, mannosidase, sialidase, and sphingomyelinase. The genetic construct may also contain one or more nucleic acid sequences encoding an activator protein (AGA) of the metabolic or lysosomal enzyme, for instance, acid alpha glucosidase (GAA) or α-galactosidase A (GALA). The genetic construct containing a nucleic acid sequence encoding a metabolic or lysosomal enzyme or a fragment or variant thereof may be suitable for or adapted for transfecting a plant cell or a plant tissue. The plant may be, for instance, a tobacco plant. The genetic construct may further contain one or more regulatory sequences, such as, for instance a promoter, enhancer or termination sequence, and may in some instances by adapted for transient or constitutive expression. An exemplary genetic construct is provided in FIG. 1.

In a seventh aspect, the invention provides a kit containing a recombinant metabolic or lysosomal enzyme or a fragment or a variant thereof with instructions or labels. The kit may be used for treating a disease caused by deficiency of a metabolic or lysosomal enzyme such as, for instance, a glycogen storage disease type II (GSDII) or acid maltase deficiency (AMD) or Pompe disease or Fabry disease. The recombinant metabolic or lysosomal enzyme or a fragment or variant thereof may be present alone, may be present in a plant extract, may be in a substantially purified or isolated form, or may be in a suitable pharmaceutical composition.

In an eighth aspect, the invention provides a kit containing at least one recombinant plant cell or plant tissue or seed containing a genetic construct such as a vector containing a nucleic acid sequence encoding a metabolic or lysosomal enzyme or a fragment or variant thereof. The nucleic acid sequence may be a cDNA and may encode acid alpha glucosidase (GAA) or α-galactosidase A (GALA). Also, the nucleic acid sequence may be a cDNA and may encode one of α-N-acetylgalactosaminidase, acid lipase, aryl sulfatase A, aspartylglycosaminidase, ceramidase, α-fucosidase, β-galactosidase, galactosylceramidase, glucocerebrosidase, β-glucuronidase, heparin N-sulfatase, β-hexosaminidase, iduronate sulfatase, α-L-iduronidase, α-mannosidase, β-mannosidase, sialidase, and sphingomyelinase. The genetic construct may also contain one or more nucleic acid sequences encoding an activator protein (AGA) of the metabolic or lysosomal enzyme, for instance, acid alpha glucosidase (GAA) or α-galactosidase A (GALA). The genetic construct containing a nucleic acid sequence encoding a metabolic or lysosomal enzyme or a fragment or variant thereof may be suitable for or adapted for transfecting a plant cell or a plant tissue. The plant may be, for instance, a tobacco plant. The genetic construct may further contain one or more regulatory sequences, such as, for instance a promoter, enhancer or termination sequence, and may in some instances by adapted for transient or constitutive expression. An exemplary genetic construct is provided in FIG. 1.

### BRIEF DESCRIPTION OF THE FIGURES

FIG. 1 is a diagram of the plant vector pBI101-CONG-GAA containing the location of the human GAA cDNA and other elements needed for expression in tobacco seeds.

FIG. 2 is a graph demonstrating uptake of tobrhGAA and placental GAA by GSDII fibroblast cells (mean±SD). Varying amounts of crude extract of seeds (equivalent to 1, 2 and 4 tobrhGAA) or 2.5, 5 and 10 μg purified human placental GAA (positive control) were added to 10⁶ human GSDII fibroblast cells. At 6 hours, cells exposed to either source of GAA had increased activity which increased as the amount of GAA was increased. The internalized tobrhGAA reversed the enzymatic defect in the fibroblasts to approximately 40% of normal GAA activity.

FIG. 3 is a graph demonstrating uptake of tobrhGAA and placental GAA in white blood cells (WBCs) from adult GSDII whole blood (mean±SD). A crude extract of tobrhGAA seeds (100 mg or ˜25 μg tobrhGAA) or placental GAA (4 μg) or mock treated with PBS to 3×3 ml heparinized whole blood from an adult onset patient, incubated samples with rocking at 37° C. for 24 hours and isolated WBCs by hypaque-ficoll density centrifugation. WBCs cells mock treated with PBS had a relative GAA activity of 5 (mean±1). Cells treated with the tobrhGAA had a relative GAA activity of 24 (mean±6) while cells treated with placental GAA had a relative GAA activity of 35 (mean±7). Students t-test comparison between mock versus tobrhGAA treated cells was p<0.007, mock versus placental GAA was p<0.0003, and p<0.02 for tobrhGAA versus placental GAA.

FIG. 4 represents Sephadex G100 chromatography of tobrhGAA. Transgenic seeds #3 were homogenized and applied the supernatant to a Sephadex G100 column. The matrix was washed until no proteins were detected by A₂₈₀ and the bound tobrhGAA eluted in buffer containing 0.25% maltose.

FIG. 5 demonstrates recovery in grip strength after oral-ERT tobrhGAA seeds were provided to subjects. Fore-limb grip strength was measured. Wild-type mice average 245±21 lbs. (SEM) grip at release. Mock treated GAA KO mice average 92±3 lbs. grip at release, and treated GAA KO mice average 105±3 lbs. grip at release. Treated GAA KO mice showed a 14% improvement in fore-limb grip strength.

FIG. 6 provides a Western blot for hGALA showing the 49 kD band in transgenic tobacco seeds number 1, 2, 4 and 5 and is not detected in wild-type (wt) seeds.

### DETAILED DESCRIPTION OF THE INVENTION

Before the present methods are described, it is to be understood that this invention is not limited to particular methods and experimental conditions described, as such methods and conditions may vary. It is also to be understood that the terminology used herein is for purposes of describing particular embodiments only, and is not intended to be limiting, since the scope of the present invention will be limited only by the appended claims. As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth in their entirety.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entireties.

### DEFINITIONS

The terms used herein have the meanings recognized and known to those of skill in the art, however, for convenience and completeness, particular terms and their meanings are set forth below.

“Subject” or “patient” refers to a mammal, preferably a human, in need of enzyme replacement therapy.

By “fragment thereof” is meant a portion of a full length protein or peptide, for instance, about 25%, 30%, 40%, 50%, 60%, 70%, 80%, 90%, 95% or more as many amino acids as the full length naturally occurring protein or peptide. By “variant thereof” is meant a fragment or full length protein or peptide, having about 50%, 60%, 70%, 80%, 90%, or 95% or more sequence homology to a corresponding naturally occurring protein or peptide. Those of skill in the art readily understand that some amino acids may be substituted without substantially impacting biological activity of a protein or peptide.

“Treatment” or “treating” refers to therapy, prevention and prophylaxis and particularly refers to the administration of medicine or performing medical procedures with respect to a patient, for either prophylaxis (prevention) or to cure or reduce the extent of or likelihood of occurrence of the infirmity or malady or condition or event. In the present invention, the treatments using the agents described may be provided to treat a glycogen storage disease. Most preferably, treatment is for the purpose of reducing or diminishing the symptoms or progression of a disease or disorder of glycogen storage. Treating as used herein also means the administration of the compounds for preventing the development of a glycogen storage disease. Furthermore, in treating a subject, the compounds of the invention may be administered to a subject already suffering from the disease.

In a specific embodiment, the term “about” means within 20%, preferably within 10%, and more preferably within 5% and in some instances within 1% or less.

An “effective amount” or a “therapeutically effective amount” is an amount sufficient to decrease or prevent the symptoms associated with the conditions disclosed herein as well as an amount sufficient to slow or prevent further pathological damage. For example, an “effective amount” for therapeutic uses is the amount of the composition comprising an active compound herein required to provide a clinically significant increase in healing rates or reduction in symptoms or to reduce morphological change including glycogen deposition.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

An individual “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of a disease or condition characterized by glycogen deposition. An individual having one or more of these risk factors has a higher probability of developing a disease than an individual without these risk factors.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entireties.

In accordance with the present invention there may be employed conventional molecular biology, microbiology, and recombinant DNA techniques within the skill of the art. Such techniques are explained fully in the literature. See, e.g., Sambrook, et al., “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994)]; “Current Protocols in Immunology” Volumes I-Ill [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Seeds may be a better vehicle for Oral-ERT for lysosomal, metabolic diseases such as Pompe disease. That is, there may be benefits to oral seed delivery of large enzymes or proteins, e.g. greater than 30 kD, compared to systemic delivery. Some of those benefits include the following. First, seeds contain the metabolic machinery necessary for correct glycosylation, processing, phosphorylation and synthesis of complex enzymes and proteins not found in other plant tissues and organelles. Second, when delivered via seeds, the large enzymes or proteins are protected or shielded from digestion in the stomach and small intestine by the sacrificial carrier material in seeds. Third, when delivered via seeds, the large enzymes or proteins may be provided for daily single and multiple administrations. Fourth, seeds are a relatively large biomass and are relatively inexpensive to produce. Fifth, when delivered via seeds, the large enzymes or proteins are relatively stable long-term.

Genetically engineered tobacco seeds are an alternative large-scale production system that overcomes the barrier of the high cost of producing recombinant human enzymes for effective enzyme replacement therapy. Several expression systems have been developed in plants that potentially offer many advantages in terms of production, scaling up, economy and safety of the therapeutic molecules (Fischer, et al., Biotechnol Adv., 2012; 30: 434-9; Lico, et al., Plant Cell Rep., 2012; 31: 439-51; Twyman, et al., Trends Biotechnol., 2003; 21: 570-578; Twyman, et al., Trends Biotechnol., 2009; 27: 609-12). The technological platform involving the accumulation of recombinant proteins in seeds warrants a better availability of the products and allows long-term storage of the biomass for processing (Kusnadi, et al., Biotechnol Bioeng., 1998; 60: 44-52; Reggi, et al., Plant Molecular Biology, 2005; 57: 101-113; Stoeger, et al., Plant Molecular Biology, 2000; 42: 583-590).

The human acid alpha glucosidase (GAA) cDNA was cloned into the plant binary vector pBI121. Transgenic plants were generated by triparental mating with Agrobacterium tumenfaciens. Expression of the recombinant acid alpha glucosidase (GAA) protein was examined in the chloroplasts, callus and leaves of transgenic tomato and tobacco and verified by immunological techniques (Western or rocket immunoelectrophoresis). Despite protein expression in the chloroplasts, callus and leaves of transgenic tomato and tobacco, the recombinant acid alpha glucosidase (GAA) protein was not enzymatically active as determined by assaying with 4-methylumbellifery-α-D-glucoside at pH 4.0 (Martiniuk, et al., Arch Biochem Biophys., 1984; 231: 454-60). Since tobacco seeds contain the metabolic machinery that is more compatible with mammalian glycosylation-phosphorylation and processing, it is preferred to produce a functional GAA protein in tobacco seeds.

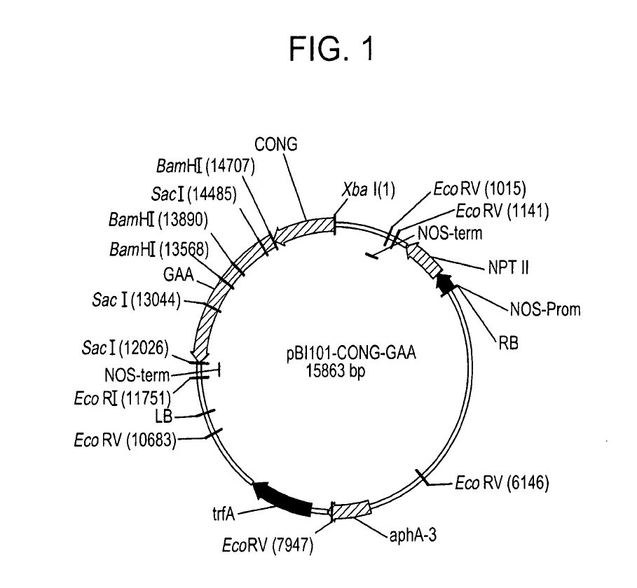

Initially, the human GAA cDNA was sub-cloned into the plant binary vector pBI121. Transgenic plants were generated by triparental mating with Agrobacterium tumenfacien. Expression of the recombinant GAA protein was examined in the chloroplasts, callus and leaves of transgenic tomato and tobacco and verified by immunological techniques (Western or rocket immunoelectrophoresis). Despite protein expression in the chloroplasts, callus and leaves of transgenic tomato and tobacco, the recombinant GAA protein was not enzymatically active as determined by assaying with 4-methylumbellifery-α-D-glucoside at pH 4.0. To generate a functional recombinant human acid alpha glucosidase (GAA) enzyme in tobacco seeds (tobrhGAA) for enzyme replacement therapy, the human acid alpha glucosidase (GAA) cDNA was subcloned into the plant expression plasmid-pBI101 under the control of the soybean β-conglycinin seed-specific promoter (FIG. 1) and biochemically analyzed the tobrhGAA. The tobrhGAA was enzymatically active and was readily taken up by GSDII fibroblasts and in white blood cells (WBCs) to reverse the defect. Additionally, the tobrhGAA could be purified since it bound tightly to the matrix of Sephadex G100 and could be eluted by competition with maltose. These data demonstrate that the tobrhGAA which is predominantly proteolytically cleaved and contains the minimal phosphorylation and mannose-6-phosphate residues retains biological activity.

In Vivo Studies in GAA^{-/-} Mice

To further evaluate if the tobrhGAA can reverse the enzyme defect in tissues, a lysate from 300 mg (˜75 μg tobrhGAA) transgenic seeds was administered intraperitoneally (IP) to five GAA knockout (GAA^{-/-} exon 6^{neo}) mice. At day 7, mice were sacrificed and tissues were assayed for activities of GAA and neutral alpha-glucosidase (NAG) and compared to normal and mock treated GAA^{-/-} mice (Table 1). Substantial increases in GAA activity in tissues, most notably heart, skeletal muscle and diaphragm were found from GAA^{-/-} mice treated with the tobrhGAA compared to mice mock treated with PBS (mean±SD). These levels were between 10-20% of wild-type GAA activity in tissues. The tobrhGAA corrected the enzyme defect in tissues at 7 days after a single dose following intraperitoneal (IP) administration in GAA^{-/-} mice (Table 1).

TABLE 1
GAA/NAG assay of mouse tissues after administration
of tobrhGAA via intraperitoneal (IP) injection.

| | GAA/NAG (mean ± SD) | | | |
|---|---|---|---|---|
| | Skeletal Muscle | Heart | Diaphragm | Liver |
| Treated GAA^{-/-} | 0.14 ± 0.02 | 0.10 ± 0.03 | 0.21 ± 0.04 | 0.21 ± 0.04 |
| Mock GAA^{-/-} | 0.048 ± 0.003 | 0.05 ± 0.006 | 0.08 ± 0.06 | 0.047 ± 0.006 |
| Wild-type BALB/c | 1.43 ± 0.23 | 0.49 ± 0.12 | 0.86 ± 0.1 | 1.10 ± 0.18 |

Raclin, et al., Biochem J., 1989; 264: 845-849 described a heat stable protein isolated from bovine spleen and guinea pig liver that enhanced the activity of GAA. The activator protein (AGA) was partially purified by chromatography with gel-permeation and had an apparent molecular of Mᵣ 20,000-24,000 kD. Rat tissues and human urine were also found to contain AGA. AGA was identified and characterized from human urine. The human AGA has been found to increase the activity of human acid alpha glucosidase activity to at least 10-fold, relative to the activity of GAA in the absence of the activator protein. The human AGA has an approximate molecular weight of 25-30 kD and is found to be heat stable. In addition, the AGA is found to have an extended shelf life without significant loss of ability to activate GAA. In addition to GAA, AGA can enhance the enzymatic activity of non-lysosomal enzymes such as β-fucosidase, β-lactase and β-galactosidase, nine-fold, six-fold and five-fold, respectively, for breakdown of their respective substrate protein. Hence, AGA may be utilized to enhance enzymatic activity of a variety of genetic disease deficient in these enzymes (Martiniuk, U.S. Patent Publication 2001/0027250 “Activator protein of human acid maltase and uses thereof”).

In Vivo Studies in GAA^{-/-} Mice Demonstrating Oral Delivery of tobrhGAA from Seeds.

To provide a more effective and less expensive alternative enzyme replacement therapy, the efficacy of a recombinant human GAA (tobrhGAA) produced in tobacco seeds administered orally for enzyme replacement therapy of Pompe disease was determined. To evaluate if the tobrhGAA can reverse the enzyme defect in tissues after oral administration, a lysate from 300 mg (contains ˜75 μg tobrhGAA) transgenic seeds was orally gavaged to GAA^{-/-} mice (n=3) at day 1, 3 and 5. Seed (300 mg) were homogenized in mortar with pestle in the presence of extraction buffer (10 mM sodium phosphate pH 7.5). Samples were incubated in ice for 1 hour under gentle agitation and eventually centrifuged at 14,000 g for 10 minutes. At day 7, mice were sacrificed and tissues were assayed for activities of GAA and neutral alpha-glucosidase (NAG) and compared to normal and mock treated GAA^{-/-} mice (Table 2). Similar to intraperitoneal (IP) administration (Table 1), substantial increases in GAA activity in tissues, most notably heart, skeletal muscle and diaphragm were found from GAA^{-/-} mice treated with the tobrhGAA compared to mice mock treated with PBS (mean±SD). The tobrhGAA corrected the enzyme defect in tissues at 7 days after oral administration in GAA^{-/-} mice.

TABLE 2
GAA/NAG assay of mouse tissues after administration
of tobrhGAA via oral gavage.

| | GAA/NAG (mean ± SD) | | | |
|---|---|---|---|---|
| | Skeletal Muscle | Heart | Diaphragm | Liver |
| Treated GAA^{-/-}-orally | 0.12 ± 0.04 | 0.10 ± 0.06 | 0.13 ± 0.09 | 0.19 ± 0.02 |
| Mock GAA^{-/-} | 0.043 ± 0.006 | 0.06 ± 0.01 | 0.07 ± 0.03 | 0.05 ± 0.009 |
| Wild-type BALB/c | 1.50 ± 0.2 | 0.43 ± 0.19 | 0.77 ± 0.15 | 1.00 ± 0.13 |

### Pharmaceutical Compositions

The tobrhGAA and/or the tobrhAGA may be administered in a time-release/slow-release formulation (e.g. a time release matrix, a microencapsulated formulation, and the like). The pharmaceutical formulation may be a unit dosage formulation, e.g., for oral administration. Elevated serum half-life may be maintained by the use of sustained-release/time-release protein “packaging” systems. Such sustained release systems are well known to those of skill in the art. In one embodiment, the ProLease biodegradable microsphere delivery system for proteins and peptides (Tracy, Biotechnol Prog., 1998; 14: 108-15; Johnson, et al., Nat Med., 1996; 2: 795-9; Herbert, et al., Pharm Res., 1998; 15: 357-61)—a dry powder composed of biodegradable polymeric microspheres containing the protein in a polymer matrix that can be compounded as a dry formulation with or without other agents. Encapsulation may be achieved at low temperatures (e.g., −40° C.). During encapsulation, the protein(s) may be maintained in the solid state in the absence of water, thus minimizing water-induced conformational mobility of the protein, preventing protein degradation reactions that include water as a reactant and avoiding organic aqueous interfaces where proteins may undergo denaturation. One suitable process uses solvents in which most proteins are insoluble, thus yielding high encapsulation efficiencies (e.g., greater than 95%).

The invention provides methods of treatment by administering to a subject an effective amount of an enzyme of the invention. In a preferred aspect, the enzyme is recombinant or is substantially purified (e.g., substantially free from substances that limit its effect or produce undesired side-effects). The subject is preferably an animal, including but not limited to monkeys, cows, pigs, horses, chickens, cats, dogs, etc., and is preferably a mammal, and most preferably human. In one specific embodiment, a non-human mammal is the subject. In another specific embodiment, a human mammal is the subject. Accordingly, the enzymes described herein may be formulated as pharmaceutical compositions to be used for prophylaxis or therapeutic use to treat these patients.

Various delivery systems are known and can be used to administer an enzyme of the invention, e.g., encapsulation in liposomes, microparticles, or microcapsules. Methods of introduction can be enteral or parenteral and include but are not limited to intradernal, intramuscular, intraperitoneal, intravenous, subcutaneous, intranasal, epidural, topical and oral routes. The enzymes may be administered together with other biologically active agents. Administration can be systemic or local.

Such compositions comprise a therapeutically effective amount of an enzyme, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the enzyme, preferably in recombinant or purified form, together with a suitable amount of a carrier so as to provide the form for proper administration to the subject. The formulation will suit the mode of administration.

In some embodiments, the enzyme can be delivered in a vesicle, in particular a liposome (See, e.g. Langer (1990) Science 249:1527-1533; Treat, et al., in Liposomes in the Therapy of Infectious Disease and Cancer, Lopez-Berestein and Fidler (eds.), Liss, New York, pp. 353-365 (1989); pp. 317-327). In yet other embodiments, the enzyme can be delivered in a controlled or sustained release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. (1983) Macromol. Sci. Rev. Macromol. Chem. 23:61; Levy, et al. (1985) Science 228:190; During, et al. (1989)Ann. Neurol. 25:351; Howard, et al. (1989)J. Neurosurg. 71:105). Other suitable controlled release systems are discussed in the review by Langer (1990) Science 249:1527-1533.

Administration of the compositions of the present invention may be pharmacokinetically and pharmacodynamically controlled by calibrating various parameters of administration, including the frequency, dosage, duration mode and route of administration. Variations in the dosage, duration and mode of administration may also be manipulated to produce the activity required. Precise amounts of enzyme required to be administered depend on the judgment of the practitioner and are peculiar to each individual. However, suitable dosages to achieve the desired therapeutic effect in vivo may range from about 0.1 mg/kg body weight per day to about 200 mg/kg body weight per day, or from about 1.0 mg/kg body weight per day to about 100 mg/kg body weight per day, preferably about 25 mg/kg body weight per day to about 50 mg/kg body weight per day. The preferred dose will depend on the route of administration. However, dosage levels are highly dependent on the nature of the disease or situation, the condition of the subject, the judgment of the practitioner, and the frequency and mode of administration. If the oral route is employed, the absorption of the substance will be a factor effecting bioavailability. A low absorption will have the effect that in the gastrointestinal tract higher concentrations, and thus higher dosages, will be necessary. Suitable regimes for initial administration and further administration are also variable, but are typified by an initial administration followed by repeated doses at one or more hour intervals by a subsequent injection or other administration. Alternatively, continuous intravenous infusion sufficient to maintain desired concentrations, e.g. in the blood, are contemplated. The composition may be administered as a single dose multiple doses or over an established period of time in an infusion.

Appropriate dosage of the enzyme should suitably be assessed by performing animal model tests, wherein the effective dose level (e.g. ED₅₀) and the toxic dose level (e.g. TD₅₀) as well as the lethal dose level (e.g. LD₅₀ or LD₁₀) are established in suitable and acceptable animal models. Further, if a substance has proven efficient in such animal tests, controlled clinical trials should be performed. The enzymes of the present invention may be modified or formulated for administration at the site of pathology. Such modification may include, for instance, formulation which facilitate or prolong the half-life of the compound or composition, particularly in the environment. Additionally, such modification may include the formulation of a compound or composition to include a targeting protein or sequence which facilitates or enhances the uptake of the enzyme.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be orally administered in any orally acceptable dosage form including, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLES

The following examples are set forth to provide those of ordinary skill in the art with a description of how to make and use the methods and compositions of the invention, and are not intended to limit the scope thereof. Efforts have been made to insure accuracy of numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Materials and Methods

RNA Extraction, cDNA Amplification and Cloning.

Total RNA was extracted from 200 mg of human placenta with TRIzol Reagent (Life Technologies) and poly(A)⁺ fraction isolated with the polyATract mRNA Isolation System (Promega) and reverse transcribed with M-MLV enzyme (Stratagene) using specific primers for the human GAA coding sequence (GAT ATC CTA ACA CCA GCT GAC GAG AAA CTG). Amplification of the GAA coding sequence was performed by combining the reverse primer with a second forward primer (GAT ATC TGC ACA CCC CGG CCG TCC CAG) matching the 5′ terminus of the cDNA sequence (GenBank Acc. No. Y00839). An EcoRV site was inserted respectively in the forward and in the reverse primer to facilitate subsequent cDNA cloning in the plant expression vector. The cDNA for mature GAA was cloned under the control of the soybean β-conglycinin promoter (GenBank Acc. No. M13759). The seed-specific promoter together with the relative 5′ UTR and transit peptide sequence was amplified from soybean DNA with primers inserting an XbaI and BamHI site (forward primer: TCT AGA GTT TTC AAA TTT GAA TTT TAA TGT GTG TTG and reverse primer: GGA TCC CAC CTT AAG GAG GTT GCA ACG AGC GTG GCA). Controlling elements and mature peptide sequence were assembled in pUC19 (Pharmacia-Amersham) and the whole tract cloned in pBI101 (Clontech) in place of the gusA gene.

Tobacco Transformation and Molecular Analysis of Transgenic Plants.

The engineered plasmids were introduced in Agrobacterium tumefaciens strain EHA105 by electroporation. Tobacco leaf discs (Nicotiana tabacum L., cv. Xanthi) were transformed as described previously (Horsch, et al, Science, 1985; 227: 1229-1231). Putatively transformed (kanamycin-resistant) plants were potted in peat and hardened in a greenhouse together with controls (plants of the donor cultivar raised in vitro from uninfected discs). Total genomic DNAs were isolated from leaves of putative transgenic and wild-type tobacco plants as described by Doyle and Doyle (Doyle, et al., Phytochem Bull., 1997; 19: 11-15) and evaluated by specific PCR. Genomic DNA of transgenic plants was extracted and PCR amplification to detect the GAA gene was carried out using primers specific for the human GAA coding sequence. Cycling conditions were: 94° C.×2′; 94° C.×45″; 58° C.×45″; 72° C.×2′ for 40 cycles with a final 72° C.×5′.

Protein Extraction.

Seed samples (100 mg) were homogenized in a mortar with pestle in the presence of 1 ml of extraction buffer (50 mM Tris, 5 mM EDTA, 200 mM NaCl, 0.1% Tween 20, pH 8.0 and 10 mM PMSF). Samples were incubated on ice for 1 hour under gentle agitation and eventually centrifuged at 14,000 g for 10 minutes. The supernatants were recovered and assayed for GAA using the artificial substrate 4-methylumbelliferyl-α-D-glucoside at pH 4.0 and as an internal control, neutral alpha glucosidase (NAG) was assayed at pH 7.5.

Western Analysis.

Samples (80 μg total protein) were electrophoresed in a 10% polyacrylamide gel and transferred to a nitrocellulose membrane (Hybond ECL, Amersham) with the Trans-Blot apparatus (BioRad) and filters were incubated for 1 hour at room temperature with rabbit polyclonal anti-GAA serum (1:10000) (Martiniuk, et al., Arch Biochem Biophys., 1984; 231: 454-60). After incubation for 1 hour with an HRP-conjugated secondary antibody (1:10,000), chemiluminescence was developed with ECL Western Blotting Detection Reagents (Amersham).

Enzyme Assay.

GAA or NAG activity was determined using 100 μl of the artificial substrate 4-methylumbelliferyl-α-D-glucoside pH 4.0 for GAA or pH 7.5 for NAG for 2-24 hours and fluorescence was determined in a fluorometer (excitation-360 nm and emission-460 nm) (Sequoia-Turner) as previously described (Martiniuk, et al. Biochem Biophys Acta., 1981; 658: 248-261).

Purification of the tobrhGAA.

Seeds were lysed as described above and clarified by centrifugation. The supernatant was adjusted to 1 mM EDTA, 25 mM sodium chloride pH 5.0 at 4° C. and applied to a Sephadex G100 column (Amersham Pharmacia Biotech Inc.) (Martiniuk, et al., Arch Biochem Biophys., 1984; 231: 454-60). The matrix was washed until no proteins were detected and the bound tobrhGAA was eluted in buffer containing 0.25% maltose.

Uptake of tobrhGAA by GSD II Human Fibroblast Cells and Peripheral Blood Lymphocytes.

Varying amounts of tobrhGAA (as crude extract equivalent to 1, 2 and 4 μg of tobrhGAA) were added to 10⁶ SV40-Ad5 immortalized human GAA-deficient fibroblast cells (TR4912) in 10% fetal bovine serum, DMEM (Life Technologies) (Martiniuk, et at, Biochem Biophys Res Comm., 2000; 276: 917-923). Cells were harvested after various hours of exposure to the exogenous GAA, washed with PBS, lysed by sonication and assayed for human GAA and NAG as described above.

Ex Vivo Experiments.

A crude extract of tobrhGAA from 100 mg of seeds or placental GAA (4 μg) or mock-treated with PBS was added to 3×3 ml heparinized whole blood from an adult onset patient, incubated samples at 37° C. for 24 hrs on a rocker and WBCs were isolated with Accu-Prep (Accurate Chemical and Scientific Corp.). Cells were assayed for GAA and NAG.

In vivo studies in the GAA^{-/-} mice.

The GAA^{-/-} mouse with the exon 6^{neo} disruption, wild-type BALB/c or GAA^{-/-} mice mock-treated with PBS were used. Five GAA^{-/-} mice (˜4 months old-males) were intraperitoneally infused with a single dose of lysate from 300 mg (˜75 μg tobrhGAA) of transgenic seeds. At 7 days, mice were sacrificed and tissues were assayed for GAA and NAG and compared to wild-type mice and mock (PBS) treated GAA^{-/-} mice.

Results. The recombinant human GAA was accumulated in the mature seed of tobacco, where recombinant proteins are stored in high quantity and stably maintain their enzymatic activity even after several months at room temperature. For promoter, the gene coding for soybean β-conglycinin, a seed protein synthesized in very large. The expression of β-conglycinin is highly regulated, being restricted to the embryo during the mid-maturation phase of embryogenesis. The promoter sequence was amplified from genomic DNA of the soybean (Glycine max Merr.) and subcloned. To target the human GAA into the endoplasmic reticulum, the signal peptide sequence of soybean β-conglycin was used in place of the native signal to allow proper processing and translocation (Reggi, et al., Plant Molecular Biology, 2005; 57: 101-113). Therefore, promoter, 5′-UTR and shuttle peptide sequence were ligated upstream of the human GAA cDNA (FIG. 1). After mobilization of the engineered vector (pBI101-CONG-GAA) to A. tumefaciens EHA105, tobacco (N. tabacum, cv. Xanthi), transformation was carried out according to standard procedures (Horsch, et al., Science, 1985; 227: 1229-1231). Several shoots (40 independently-transformed plants) survived levels of kanamycin (selective agent) as high as 100 μg/l. Molecular analysis confirmed the integration of the transgene in 87% of transgenic plants. Western analysis demonstrated the tissue-specific expression of recombinant human GAA and its accumulation in developing seeds in 66% of PCR-positive plants. The antibody reacted with two major bands of 80 and 70 kD, having an apparent molecular weight very similar to human placenta. No cross-reacting proteins were identified in wild-type seed extracts nor traces of degradation products in any transformed sample (data not shown).

Protein Extraction and Assay for GAA.

One hundred mg of seeds from transgenic plants were homogenized and the supernatants assayed for GAA and as an internal control, neutral alpha maltase (NAG) was assayed at pH 7.5. Wild-type tobacco seeds had a GAA/NAG ratio of 0.05 while seeds from transgenic plants ranged from 0.1 to 2.0. Transgenic plant #3 had the greatest activity, estimated to contain 25 μg tobrhGAA/100 mg or 250 μg/g seeds. This extract was frozen and thawed 4× over 2 weeks without losing any substantial GAA activity.

Western Analysis.

Extracts from seeds #3 were analyzed by Western analysis on a 10% SDS-PAGE with rabbit polyclonal anti-placental GAA serum (data not shown). These data demonstrate that the tobrhGAA was similar to native human placental GAA showing two high molecular weight bands (˜80 and 70 kD) plus a third band of 100 kD. The 100 kD band may represent proteolytically uncleaved GAA. Smaller bands of 20-25 kD were not observed.

Uptake by GSDII Fibroblasts.

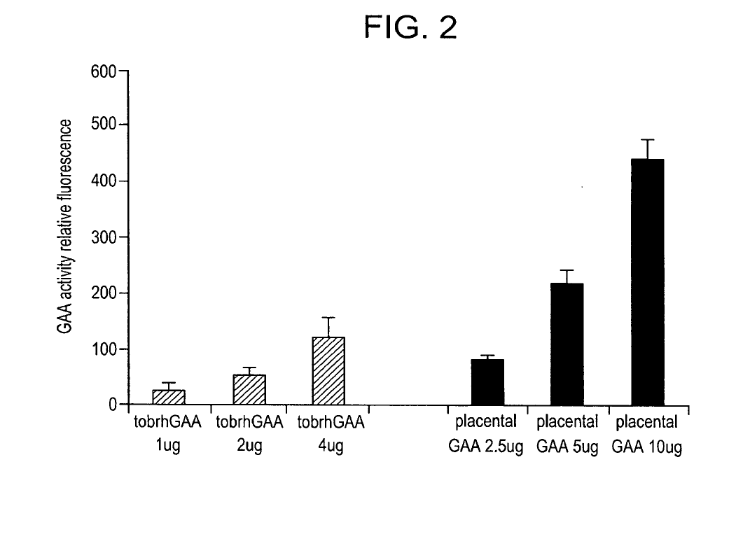

A critical experiment to evaluate the functional status of the tobrhGAA is uptake by human GSDII fibroblast cells. Varying amounts of crude extract of seeds (equivalent to 1, 2 and 4 μg tobrhGAA) or 2.5, 5 and 10 μg purified human placental GAA (positive control) were added to human GSDII fibroblast cells. At 6 hours, cells exposed to either source of GAA had increased activity which increased as the amount of GAA was increased (FIG. 2). At maximum amounts of tobrhGAA, 40% of normal GAA was observed. Finally, to evaluate the longevity of the internalized GAA, cells were exposed to a constant amount of placental GAA or tobrhGAA for 6 hrs. The media lacking any exogenous enzyme was replaced and cells were harvested after 24, 48 and 168 hour. Exposure to either GAA sources showed activity identical for 6 and 24 hours incubation (data not shown). Minimal uptake was observed when cells were pretreated with 5 mM mannose-6-phosphate (data not shown).

Ex Vivo Studies.

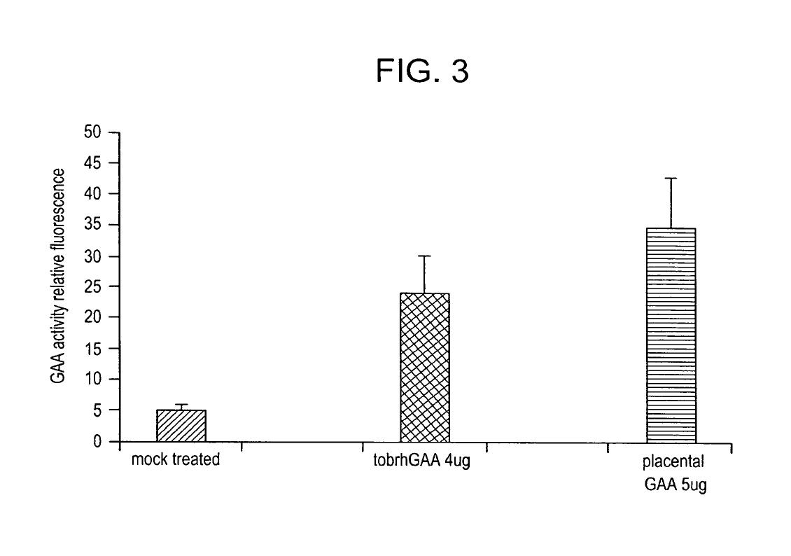

A crude extract of tobrhGAA seeds (100 mg or ˜25 μg tobrhGAA calculated from specific activity) or placental GAA (4 μg) or mock-treated (PBS) was added to white blood cells (WBCs) from whole blood from an adult onset patient. After incubation, isolated WBCs were assayed for GAA. PBS mock-treated WBCs had a relative GAA activity of 5 (mean±1); WBCs treated with the tobrhGAA had a relative GAA activity of 24 (mean±6) while WBCs treated with placental GAA had a relative GAA activity of 35 (mean±7) (FIG. 3). Students t-test comparison between mock versus tobrhGAA treated cells was p<0.007; mock versus placental GAA was p<0.0003 and p<0.02 for tobrhGAA versus placental GAA.

Purification of the tobrhGAA.

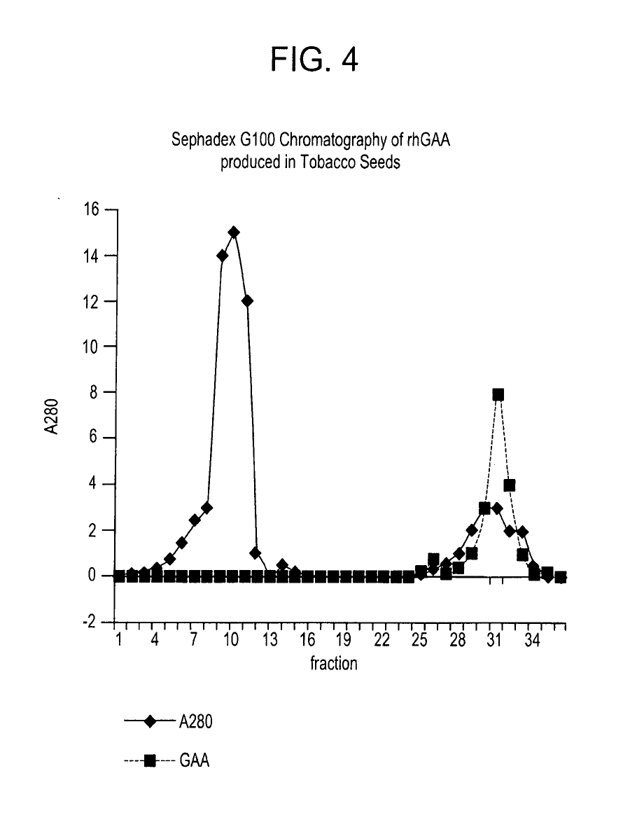

Sephadex G100 is a natural affinity matrix for the mature, fully processed, glycosylated GAA (Martiniuk, et al., Arch Biochem Biophys., 1984; 231: 454-60). If the mature enzyme is not processed and glycosylated, binding to Sephadex G100 is very weak. To determine if the tobrhGAA can bind to Sephadex G100 (important for future large scale purification), homogenized were seeds and the supernatant was applied to a Sephadex G100 column. The matrix was washed until no proteins were detected by A₂₈₀ and the bound tobrhGAA was eluted in buffer containing 0.25% maltose (FIG. 4). The specific GAA activity of the bound and eluted tobrhGAA was 8,000 IU/g as compared to purified human placental GAA of 12,000-15,000 IU/g as determined by enzyme assay. Recovery was approximately 15%.

In Vivo Studies in GAA^{-/-} Mice.

To evaluate if the tobrhGAA can reverse the enzyme defect in tissues, a lysate from 300 mg (˜75 μg tobrhGAA) transgenic seeds was administered intraperitoneally (IP) to five GAA^{-/-} mice (exon 6^{neo}). At day 7, mice were sacrificed and tissues were assayed for GAA and NAG and compared to wild-type and mock-treated GAA^{-/-} mice (Table 1). There were substantial increases in GAA activity in tissues, most notably in heart, skeletal muscle and diaphragm from GAA^{-/-} mice treated with the tobrhGAA compared to mice mock-treated with PBS (mean±SD). These levels were between 10-20% of wild-type GAA activity in tissues. To provide a more effective and less expensive alternative enzyme replacement therapy, the efficacy of a recombinant human GAA (tobrhGAA) produced in tobacco seeds administered orally for enzyme replacement therapy of Pompe disease was determined. To evaluate if the tobrhGAA can reverse the enzyme defect in tissues after oral administration, a lysate from 300 mg (contains ˜75 μg tobrhGAA) transgenic seeds was orally gavaged to GAA^{-/-} mice (n=3) at day 1, 3 and 5. Seed (300 mg) were homogenized in mortar with pestle in the presence of extraction buffer (10 mM sodium phosphate pH 7.5). Samples were incubated in ice for 1 hour under gentle agitation and eventually centrifuged at 14,000 g for 10 minutes. At day 7, mice were sacrificed and tissues were assayed for activities of GAA and neutral alpha-glucosidase (NAG) and compared to normal and mock treated GAA^{-/-} mice (Table 2). Similar to intraperitoneal (IP) administration (Table 1), substantial increases in GAA activity in tissues, most notably heart, skeletal muscle and diaphragm were found from GAA^{-/-} mice treated with the tobrhGAA compared to mice mock treated with PBS (mean±SD). The tobrhGAA corrected the enzyme defect in tissues at 7 days after oral administration in GAA^{-/-} mice.

Discussion

Currently, there is no effective treatment or cure for GSDII. Lysosomal enzymes (such as GAA) are targeted to the lysosome by a mannose-6-phosphate recognition sequence that is exposed by posttranslational modification in the Golgi that may be the mechanism that extracellular GAA can be recycled and targeted back to the lysosomes. This mechanism potentially allows recombinant human GAA to be delivered to the cells or tissues and directed to the lysosome. However, some GAA may be taken up or recycled by endocytosis or a mannose-6-phosphate independent mechanism (Bijvoet, et al., Hum Mol Genet., 1998; 1815-24; Maga, et al., J Biol Chem., 2013; 288: 1428-38; Van der Ploeg, et al., J Neurol., 1988; 235; 392-6; Van der Ploeg, et al., J Clin Invest., 1991; 87: 513-8). A number of groups have tried to mass produce a recombinant human GAA (rhGAA). One group started Phase I/II trials with a recombinant human GAA secreted into rabbit milk (Van den Hout, et al., Pediatrics, 2004; 113: 448-457). Although promising, their rhGAA was not successful in treating patients. Another group using a rhGAA secreted from a CHO cell line has demonstrated moderate success in patients (Kikuchi, et al., J Clin Lab., 1998; 101: 827-33), however yearly costs are very high. Thus, to provide a less expensive alternative, work was started to generate and evaluate a recombinant human GAA produced in tobacco seeds for enzyme replacement therapy of AMD. Tobacco seeds contain metabolic machinery that is more compatible with mammalian glycosylation-phosphorylation and processing. There have been a number of enzymes or proteins produced in seeds including human collagen type α-1 in maize seeds (Xu, et al., BMC Biotechnol., 2011; 11: 69-80), human lysosomal α-mannosidase (MAN2B1) in Nicotiana benthamiana leaves and seeds (De Marchis, et al., Plant Biotechnol J., 2011; 9: 1061-73), Ascaris suum As14 protein and its fusion with cholera toxin B subunit in rice seeds (Nozoye, et al., J Vet Med Sci., 2009; 71: 995-1000), cholera toxin B subunit in transgenic rice endosperm (Oszvald, et al., Mol Biotechnol., 2008; 40: 261-8), human CD14 in tobacco seeds (Blais, et al., Transgenic Res., 2006; 15: 151-64), human lactoferrin in maize and tobacco (Samyn-Petit, et al., Eur J Biochem., 2003; 270: 3235-42) and maize (Zea mays)-derived bovine trypsin characterization for large-scale, commercial product from transgenic plants (Woodard, et al., Biotechnol Appl Biochem., 2003; 38: 123-30). The tobrhGAA was enzymatically active and was readily taken up by GSDII fibroblasts. In WBCs from whole blood, the tobrhGAA corrected the enzyme defect in tissues at 7 days after a single intraperitoneal (IP) administration in GAA^{-/-} mice. Additionally, the tobrhGAA could be easily purified because it bound tightly to the matrix of Sephadex G100 and could be eluted by competition with maltose. These data demonstrate indirectly that the tobrhGAA is fully functional, proteolytically cleaved and contains the minimal phosphorylation and mannose-6-phosphate residues to maintain activity. Only the native, fully processed human GAA binds tightly to Sephadex G100. Data in E. coli (Martiniuk, et al., DNA Cell Biol., 1992; 11: 701-6) and unpublished data in yeast have found that the recombinant human GAA from both systems (that may have altered glycosylation/processing) show substantially reduced GAA activity despite the GAA mRNA being highly expressed. Additionally, the purified tobrhGAA has high specific activity, similar to the native human placental GAA making it ideal for enzyme replacement therapy. Estimates on production are: 200 flowers per plant; about 1,300 seeds per flower and 1,000 seeds weighs 0.1 grams, thus 26 grams of seeds per plant. There are about 24,000 plants per acre or 60,000 per hectare. A hectare can produce about 1,444 kg of seeds. Data suggests that there are 250 μg tobrhGAA/gram seeds, or one hectare can produce 361 g of purified tobrhGAA. Hence, the cost of seed production of tobrhGAA will be much lower than rhGAA produced from CHO cells. Current cost for enzyme replacement therapy with the latter ranges from $250,000 to $650,000 per patient depending upon weight.

### Example 2

### Activator Protein (hAGA)

It is desirable to identify, clone and functionally analyze a human activator protein (hAGA) and develop a transgenic tobacco plant expressing hAGA (tobrhAGA). The gene/protein sequence of the human activator protein (AGA) may be determined by standard molecular/protein methodology and clone the gene. A recombinant hAGA may be generated in an appropriate expression system and proof of feasibility/efficacy tested by activating normal human GAA. It is possible to evaluate activation of patient mutant GAA and uptake by normal and patient cell lines (fibroblast, lymphoid and skeletal muscle) and activation of internal GAA. Various concentrations and times of exposure may be tested.

A transgenic tobacco plant expressing a recombinant human activator protein (tobrhAGA) may be generated by tri-parental mating with the plant binary vector above and determine expression in various organelles (leaves, stems, seeds, etc). A functional tobrhAGA may be localized in the leaves since post-translational modification may not be required. Proof of feasibility/efficacy of the tobrhAGA may be evaluated by activating human GAA.

GAA^{-/-} mice (n=5) will be treated orally daily vs. IP-every 2 weeks with a bolus of the tobrhGAA or Myozyme and analyzed for reversal of AMD by biochemical, clinical phenotype presentation (muscle weakness) and histology parameters. GAA^{-/-} mice will receive the tobrhGAA at four different and escalating doses. Mice will be sacrificed biweekly for two months. Tissues, urine and serum will be collected for analysis. Analysis will include GAA assay, histology, glycogen content, Western analysis, pharmacokinetics (uptake, max-Cₛ, T_{1/2} and excretion) and ELISA to evaluate increase in enzyme activity.

Oral administration of tobrhAGA+/−tobrhGAA in GAA^{-/-} mice (n=5) will be compared as described herein. The expected feasibility outcomes of combined oral administration include increase GAA activity/protein in tissues to 10% of normal, reversal of clinical phenotype, decreased glycogen greater than administration of single agents.

### Example 3

### Nicotine Levels in Leaves and Seeds

The level of nicotine in tobacco leaves and seeds was measured by thermo desorption/gas chromatography-mass spectroscopy (GC/MS) (Avogado n Analytical, LLC, Salem, N.H. 03079-2862). The nicotine level was determined to be <5 ng/dry gram of tobacco tobrhGAA seeds and leaves.

### Example 4

### Long Term Stability

To test the stability of tobrhGAA in seeds, GAA levels were measured in seeds that had been stored for 9 years at room temperature and in freshly harvested recombinant seeds from plant #3. There was less than a 10% difference in GAA activity between the two groups of seeds indicating great stability (old=0.23 mg tobrhGAA/gram seeds vs. fresh-0.25 mg tobrhGAA/gram seeds).

### Effect of Stomach and Small Intestinal Environment on Stability.

To mimic the stomach and small intestine environment, the tobrhGAA was exposed at physiologic levels, conditions and times to pepsin (as in the stomach) and trypsin/chymotrypsin (as in the small intestine). Pepsin in the stomach (pH 1-5) ranges from 50-300 μg/ml, trypsin is present at about 800 μg/ml, and chymotrypsin is present at about 700 μg/ml in the duodenum of the small intestine (pH 6-8). Food spends about 30-80 minutes in the stomach and 60-300 minutes in the small intestine.

A lysate from tobrhGAA #3 seeds was exposed to 300 μg/ml pepsin with 1 mg/ml bovine serum albumin at pH 4.0 or trypsin at 800 μg/ml at pH 6.5 and chymotrypsin at 700 μg/ml at pH 6.5 with 1 mg/ml bovine serum albumin for 60 minutes at 37° C. and then assayed for GAA activity. None of the enzymes had any effect on tobrhGAA enzyme levels thus demonstrating that the times and conditions in the digestive tract do not affect tobrhGAA.

### Example 5

### Safety and Toxicity

Safety and toxicity were investigated in wild-type mice (Swiss-Webster) orally treated 6 days a week for 2 and 4 weeks with a lysate from tobrhGAA seeds #3. In summary, there was no difference in general appearance observed between mock treated and treated mice. No significant change in weight (0%, 1%, or 2% increase or decrease in weight between groups) was observed for mock or treated mice. There was no difference in the percentage of blood smear differentials or complete blood counts for neutrophils, lymphocytes, monocytes, eosinophils and basophils between mock or treated mice at four weeks.

### Example 6

### Correction of Fore-Limb Grip Strength

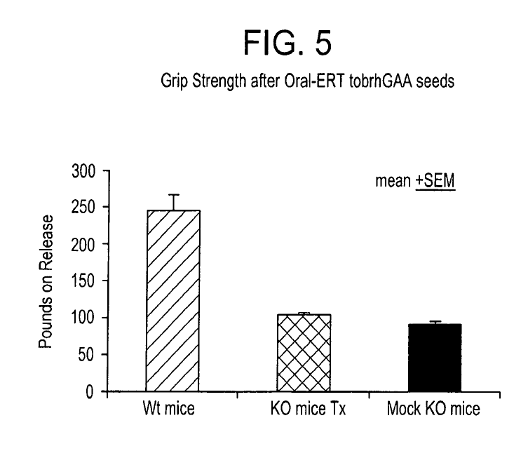

Whether it is possible to correct the disease phenotype of fore-limb muscle weakness in GAA KO mice after 3 oral administrations, every other day with either a lysate of tobrhGAA seeds #3 or mock treated was investigated. Fore-limb grip strength was measured using a grip-strength meter (Columbus Instruments). Wild-type mice (n=3) average 245+/−21 lbs. (SEM) grip at release. Mock treated GAA KO mice (n=3) average 92+/−3 lbs. grip at release (3 attempts/mouse), and treated GAA KO mice (n=3) average 105+/−3 lbs. grip at release (p<0.024 treated vs. mock treated GAA KO mice) (FIG. 5). Treated GAA KO mice showed a 14% improvement in fore-limb grip strength.

### Example 7

### An Enzyme Produced in Recombinant Transgenic Seeds Useful for Treating Fabry Disease

Transgenic tobacco plants were generated expressing the human gene for α-galactosidase A (GALA—NCBI Reference Sequence: NM_000169.2) using the identical cloning strategy, promoter and vector as described above (Example 1; FIG. 1). Fifteen transgenic plants potentially expressing the human GALA protein in tobacco seeds (tobrhGALA) were generated. Lysate from the recombinant seeds was analyzed with 4-MUF-α-D-galactopyranoside (Fisher 50-213-471)(2 mg/ml) in 0.1 M sodium citrate pH 4.5 at 37° C. More than one-half of the plants had increased activity over wild-type seeds. The maximum activity reported was 0.7 U/gram seeds.

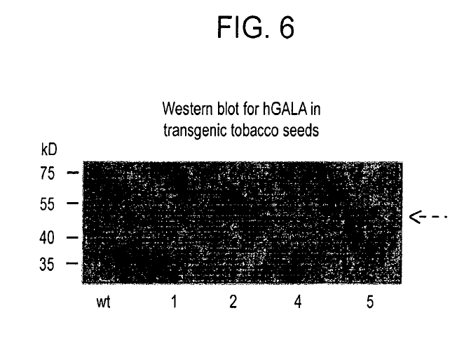

To confirm that the enzyme activity was reflected by the protein levels, Western analysis for human GALA was performed. Samples (30 μL) were electrophoresed on a 12% PAGE SDS gel (BioRad reagents) and transferred to PVDF at 100 volts for 2 to 3 hours. Unreacted sites were blocked in 5% nonfat dry milk. Primary antibody (Proteintech 49kD alpha galactosidase A rabbit polyclonal (#15428-1-AP) at 1:1000 (overnight) was introduced and the samples filter washed. A secondary antibody 1:2000 was added for 1 hour. The samples were washed and detected with ThermoScientific #3408 SuperSignal West Pico Chemiluminescent substrate for 100 seconds of exposure to X-ray film. FIG. 6 is a Western blot generated in this manner for hGALA showing the 49kD band in transgenic tobacco seeds number 1, 2, 4 and 5 and not detected in wild-type (wt) seeds.

1. A method for replacing a metabolic or lysosomal enzyme in a subject in need of the metabolic or lysosomal enzyme comprising orally administering the metabolic or lysosomal enzyme or a biologically active fragment or a variant thereof or a pharmaceutical composition containing the metabolic or lysosomal enzyme or a biologically active fragment or a variant thereof or a plant extract containing the metabolic or lysosomal enzyme or a biologically active fragment or a variant thereof to the subject.

2. The method according to claim 1 wherein the metabolic or lysosomal enzyme or a fragment or a variant thereof is produced recombinantly.

3. The method according to claim 2 wherein the metabolic or lysosomal enzyme or fragment or variant thereof is produced recombinantly in a plant cell.

4. The method according to claim 3 wherein the plant cell is a tobacco plant cell.

5. The method according to claim 3 wherein the plant cell is a tobacco seed.

6. The method according to claim 1 wherein the metabolic or lysosomal enzyme is acid alpha glucosidase (GAA).

7. The method according to claim 1 further comprising administering an activator protein or peptide operable to increase the biological activity of the metabolic or lysosomal enzyme.

8. A method of treating a disease caused by a deficiency of biological activity or amount of a metabolic or lysosomal enzyme comprising orally administering the metabolic or lysosomal enzyme or a biologically active fragment or variant thereof or a pharmaceutical composition or plant extract containing the metabolic or lysosomal enzyme or a biologically active fragment or variant thereof to a subject suffering from the disease.

9. The method according to claim 8 wherein the glycogen storage disease is selected from the group consisting of glycogen storage disease type II (GSDII), acid maltase deficiency (AMD), Pompe disease and Fabry disease.

10. The method according to claim 8 wherein the metabolic or lysosomal enzyme or a fragment or a variant thereof is produced recombinantly.

11. The method according to claim 10 wherein the metabolic or lysosomal enzyme or fragment or variant thereof is produced recombinantly in a plant cell.

12. The method according to claim 10 wherein the plant cell is a tobacco plant cell.

13. The method according to claim 10 wherein the plant cell is a tobacco seed.

14. The method according to claim 8 wherein the metabolic or lysosomal enzyme is acid alpha glucosidase (GAA).

15. The method according to claim 8 further comprising administering an activator protein or peptide operable to increase the biological activity of the metabolic or lysosomal enzyme.

16. A pharmaceutical composition comprising a metabolic or lysosomal enzyme or a fragment or a variant thereof and a suitable carrier.

17. A pharmaceutical composition according to claim 16 further comprising an activator protein or peptide of the metabolic or lysosomal enzyme.

18. A pharmaceutical composition according to claim 16 wherein the metabolic or lysosomal enzyme is acid alpha glucosidase (GAA).

19. A pharmaceutical composition according to claim 16 designed for sustained release.

20. A genetic construct comprising a nucleic acid sequence encoding acid alpha glucosidase (GAA) or a fragment or a variant thereof and at least one regulatory sequence.

21-24. (canceled)
